(19) United States
(12) Patent Application Publication
(10) Pub. No.: US 2026/0073518 A1
(43) Pub. Date: Mar. 12, 2026

(54) METHOD FOR PREDICTING OBJECT STATE BASED ON DYNAMIC IMAGE DATA AND COMPUTING DEVICE FOR PERFORMING THE SAME

(71) Applicants: THE ASAN FOUNDATION, Seoul (KR); UNIVERSITY OF ULSAN FOUNDATION FOR INDUSTRY COOPERATION, Ulsan (KR)

(72) Inventors: Jae Seung KIM, Seoul (KR); Jungsu OH, Seoul (KR); Minyoung OH, Seoul (KR); Dong Yun LEE, Seoul (KR); Seung Jun OH, Seoul (KR); Sang Ju LEE, Seoul (KR); Changhwan SUNG, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/389,431

(22) Filed: Nov. 14, 2025

Related U.S. Application Data
(63) Continuation of application No. PCT/KR2024/095818, filed on May 17, 2024.

(30) Foreign Application Priority Data
May 17, 2023 (KR) 10-2023-0063952
May 17, 2024 (KR) 10-2024-0064227

(51) Int. Cl. G06T 7/00; G16H 30/40; G16H 50/20; G16H 50/30

(52) U.S. Cl. CPC G06T 7/0012; G16H 30/40; G16H 50/20; G16H 50/30; G06T 2207/10104

(57) ABSTRACT

Provided are a dynamic image data-based object state prediction method and a computer device performing the same. The computing device includes a memory and at least one processor communicating with the memory. The processor obtains early dynamic image data corresponding to an early section after a medicine is injected into a learning object until a preset time point, learns a first prediction model for predicting first image data indicating anatomical information about the learning object corresponding to a time point earlier than a first section by using, as an input, early dynamic image data corresponding to the first section of the early section, and learns a second prediction model for predicting second image data indicating disease-specific information about the learning object corresponding to a reference time point after the early section by using, as an input, early dynamic image data corresponding to a second section of the early section.

### CROSS-REFERENCE TO RELATED APPLICATIONS

The present application is a continuation of International Patent Application No. PCT/KR2024/095818, filed on May 17, 2024, which is based upon and claims the benefit of priority to Korean Patent Application Nos. 10-2023-0063952 filed on May 17, 2023 and 10-2024-0064227 filed on May 17, 2024. The disclosures of the above-listed applications are hereby incorporated by reference herein in their entirety.

### BACKGROUND

Embodiments of the present disclosure described herein relate to a medical-related technology, and more particularly, relate to a method for predicting the state of a diagnosis subject based on dynamic image data and a computing device performing the same.

Positron Emission Tomography (PET) examination is a cutting-edge nuclear medicine imaging examination method that administers a positron-emitting radioisotope to a subject and captures the radiation emitted from the body to obtain useful diagnostic information related to metabolic changes and receptor distribution in the human body. More recently, the technology has evolved further in the form of hybrid scanners, which go beyond acquiring just PET images and also incorporate a computed tomography (CT) technology or a magnetic resonance image (MRI) technology.

Accordingly, the latest PET examination may obtain anatomical information from CT images by using a PET/CT scanner that combines a PET device and a CT device into one, thereby providing accurate location and depth information of a lesion identified in a PET image.

Typically, the PET examination is performed based on the PET image acquired after a specific time (e.g., 1 hour 30 minutes to 3 hours), when tracer specific binding and tracer nonspecific binding reach a steady state after a radioactive tracer is injected into the body, or when the difference between them is maximized, elapses.

However, PET image-based diagnosis is performed at a relatively late time point after tracer injection, thereby requiring a method for improving this.

### SUMMARY

Embodiments of the present disclosure provide a method for predicting the state of a diagnosis subject based on dynamic image data.

Problems to be solved by the present disclosure are not limited to the problems mentioned above, and other problems not mentioned will be clearly understood by those skilled in the art from the following description.

According to an embodiment, a method performed by at least one processor includes obtaining early dynamic image data corresponding to an early section after a medicine is injected into a learning object until a preset time point, learning a first prediction model for predicting first image data indicating anatomical information or blood flow information about the learning object corresponding to a time point earlier than a first section by using, as an input, early dynamic image data corresponding to the first section of the early section, and learning a second prediction model for predicting second image data indicating disease-specific information about the learning object corresponding to a reference time point after the early section by using, as an input, early dynamic image data corresponding to a second section of the early section.

The method may further include obtaining early dynamic image data corresponding to the first section after the medicine is injected into a diagnostic object, and predicting first image data indicating anatomical information or blood flow information about the diagnostic object corresponding to the earlier time point, by inputting the early dynamic image data corresponding to the first section of the diagnostic object into the learned first prediction model.

The method may further include obtaining early dynamic image data corresponding to the second section after the medicine is injected into the diagnostic object, and predicting second image data indicating disease-specific information about the diagnostic object corresponding to the reference time point, by inputting the early dynamic image data corresponding to the second section of the diagnostic object into the learned second prediction model.

The processor may obtain and process early dynamic image data corresponding to the first section and early dynamic image data corresponding to the second section based on the injection to the diagnostic object, and may obtain the processed early dynamic image data corresponding to the first section and the processed early dynamic image data corresponding to the second section, when the medicine reduced by a preset amount is injected into the diagnostic object, and may input the processed early dynamic image data corresponding to the first section into the first prediction model, and input the processed early dynamic image data corresponding to the second section into the second prediction model.

An amount of the medicine injected into the learning object may be an amount reduced from a reference amount. The first prediction model may be learned based on first label image data corresponding to the first image data. The second prediction model may be learned based on second label image data corresponding to the second image data, and each of the first label image data and the second label image data may be label image data processed according to a medicine injection amount of the learning object.

The method may further include performing spatial normalization before the early dynamic image data corresponding to the first section of the learning object and the diagnostic object is input to the first prediction model, and performing spatial normalization before the early dynamic image data corresponding to the second section of the learning object and the diagnostic object is input to the second prediction model.

The processor may set an acquisition time section of the early dynamic image data corresponding to the first section in the early section of the learning object and the diagnostic object, and may set an acquisition time section of the early dynamic image data corresponding to the second section in the early section of the learning object and the diagnostic object.

The processor may identify the early section when the medicine is injected into the diagnostic object, and may obtain the early dynamic image data corresponding to the first section and the early dynamic image data corresponding to the second section based on the identified early section.

Each of the obtained early dynamic image data, the first image data, and the second image data may be positron emission tomography (PET) image data.

According to an embodiment, a dynamic image data-based object state prediction device includes a memory, and at least one processor communicating with the memory.

The processor may obtain early dynamic image data corresponding to an early section after a medicine is injected into a learning object until a preset time point, may learn a first prediction model for predicting first image data indicating anatomical information or blood flow information about the learning object corresponding to a time point earlier than a first section by using, as an input, early dynamic image data corresponding to the first section of the early section, and may learn a second prediction model for predicting second image data indicating disease-specific information about the learning object corresponding to a reference time point after the early section by using, as an input, early dynamic image data corresponding to a second section of the early section.

The processor may obtain early dynamic image data corresponding to the first section after the medicine is injected into a diagnostic object, and may predict first image data indicating anatomical information or blood flow information about the diagnostic object corresponding to the earlier time point, by inputting the early dynamic image data corresponding to the first section of the diagnostic object into the learned first prediction model.

The processor may obtain early dynamic image data corresponding to the second section after the medicine is injected into the diagnostic object, and may predict second image data indicating disease-specific information about the diagnostic object corresponding to the reference time point, by inputting the early dynamic image data corresponding to the second section of the diagnostic object into the learned second prediction model.

The processor may obtain and process early dynamic image data corresponding to the first section and early dynamic image data corresponding to the second section based on the injection to the diagnostic object, and may obtain the processed early dynamic image data corresponding to the first section and the processed early dynamic image data corresponding to the second section, when the medicine reduced by a preset amount is injected into the diagnostic object, and may input the processed early dynamic image data corresponding to the first section into the first prediction model, and input the processed early dynamic image data corresponding to the second section into the second prediction model.

An amount of the medicine injected into the learning object may be an amount reduced from a reference amount. The first prediction model may be learned based on first label image data corresponding to the first image data. The second prediction model may be learned based on second label image data corresponding to the second image data, and each of the first label image data and the second label image data may be label image data processed according to a medicine injection amount of the learning object.

The processor may perform spatial normalization before the early dynamic image data corresponding to the first section of the learning object and the diagnostic object is input to the first prediction model, and may perform spatial normalization before the early dynamic image data corresponding to the second section of the learning object and the diagnostic object is input to the second prediction model.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is an overall schematic diagram showing a method for predicting the state of an object based on dynamic image data, according to an embodiment of the present disclosure;

FIG. 2 is a block diagram showing a configuration of a dynamic image data-based object state prediction device, according to an embodiment of the present disclosure;

FIG. 3 is a sequence diagram illustrating an object state prediction method based on dynamic image data, according to an embodiment of the present disclosure;

FIG. 4 is a diagram for describing a process of learning a first prediction model and a second prediction model based on early dynamic image data, according to an embodiment of the present disclosure;

FIG. 5 is a sequence diagram showing a method for predicting first image data indicating anatomical information or blood flow information of a diagnosis subject and second image data indicating disease-specific information, according to an embodiment of the present disclosure;

FIG. 6 is a sequence diagram showing a preprocessing process for performing spatial normalization, according to an embodiment of the present disclosure;

FIG. 7 is a diagram for describing spatial normalization performed in a preprocessing step before learning an artificial neural network model, according to an embodiment of the present disclosure;

FIG. 8 is a diagram showing changes in radiation dose over time after medicine injection, according to an embodiment of the present disclosure;

FIG. 9A is a graph for comparing label image data with delay image data generated by using a reference dose, according to an embodiment of the present disclosure. FIG. 9B is a graph for comparing label image data with delay image data generated by using a low dose, according to an embodiment of the present disclosure. FIG. 9C is a graph for comparing delay image data generated by using a reference dose with delay image data generated by using a low dose according to an embodiment of the present disclosure;

FIG. 10A illustrates analog PET data used in training and testing steps for each of Anterior Putamen (AP) and Posterior Putamen (PP), according to an embodiment of the present disclosure. FIG. 10B illustrates digital PET data used in the test step for each of the AP and the PP, according to an embodiment of the present disclosure;

FIG. 11 is a diagram for describing a method for obtaining true early image data, according to an embodiment of the present disclosure; and

FIG. 12 is a diagram for describing a method for obtaining delay image data, according to an embodiment of the present disclosure.

### DETAILED DESCRIPTION

Hereinafter, preferred embodiments of the present disclosure will be described in detail with reference to the accompanying drawings. The above and other aspects, features and advantages of the present disclosure will become apparent from embodiments to be described in detail in conjunction with the accompanying drawings. However, the present disclosure is not limited to the embodiments disclosed below, but may be implemented in various forms. The embodiments of the present disclosure are provided to make the present disclosure complete and fully inform those skilled in the art to which the present disclosure pertains of the scope of the present disclosure. The same reference numerals denote the same elements throughout the specification.

Unless otherwise defined, all terms (including technical and scientific terms) used herein have the same meaning as commonly understood by those skilled in the art to which the present disclosure pertains. Moreover, terms, such as those defined in commonly used dictionaries, should not be interpreted in an idealized or overly formal sense unless expressly so defined herein.

The terms used herein are provided to describe the embodiments but not to limit the present disclosure. In the specification, the singular forms include plural forms unless particularly mentioned. The terms “comprises” and/or “comprising” used herein do not exclude the presence or addition of one or more other components, in addition to the aforementioned components.

In this specification, a ‘computing device’ includes various devices capable of performing arithmetic processing. The ‘computing device’ may include one or more computers. For example, the computer may correspond to not only a desktop personal computer (PC) or a notebook but also a smart phone, a tablet PC, a cellular phone, a personal communication service (PCS) phone, a mobile terminal of a synchronous/asynchronous International Mobile Telecommunication-2000 (IMT-2000), a palm PC, a personal digital assistant (PDA), and the like. Moreover, the computer may also be a medical device that acquires or observes medical images. Furthermore, the computer may be a server computer that connects to various client computers.

In this specification, ‘image data’ means an image acquired by a medical image capturing device.

In this specification, the ‘medical image capturing device’ means a device used to acquire the medical image. For example, the ‘medical image capturing device’ may include a positron emission tomography image capturing device, a magnetic resonance imaging (MRI) image capturing device, etc.

In this specification, ‘delay image data’ refers to a diagnostic image acquired after a reference time and used for patient diagnosis.

In this specification, a ‘reference time’ means a period from a first time point, when a medicine (e.g., a contrast agent or a tracer) is injected into the human body, to a time point (i.e., the reference time point) when image data for diagnosing a patient's state is capable of being obtained.

In this specification, the ‘medicine’ means something injected into the body when the medical image data is captured. For example, the ‘medicine’ may refer to a contrast agent used in Magnetic Resonance Imaging (MRI) or Computed Tomography (CT), or a tracer used in Positron Emission Tomography.

In this specification, “early dynamic image data” refers to image data including a plurality of consecutive image frames. The “early dynamic image data” is acquired prior to a reference time point at which delay image data is acquired, and is thus acquired within an initial time range (e.g., a time range after a short period of time following the insertion of a contrast agent or a tracer administered at the time of image acquisition).

In this specification, the dynamic image data may mean image data excluding the early dynamic image data and the delay image data.

In detail, the dynamic image data may refer to a playback section image from a point in time when the blood flow effect begins to decrease to a preset reference time point after a medicine injection time point of a learning object included in each of pieces of image data.

In this specification, the anatomical information may be information about the structure and function of an object, and may include image data including blood flow information showing the blood flow of the object.

In this specification, disease-specific information may mean image data capable of being used to determine the state of an object when a medicine remains in a specific target location (e.g., an organ or tissue).

Hereinafter, a detailed description of a method and a program for generating an early dynamic image data-based diagnostic image according to an embodiment of the present disclosure will be described with reference to the drawings.

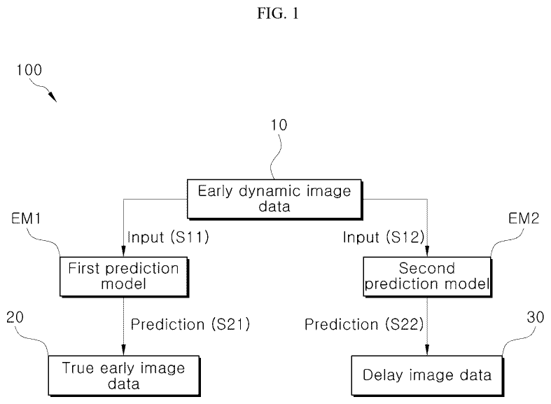

FIG. 1 is an overall schematic diagram showing a method for predicting the state of an object based on dynamic image data according to the present disclosure, and which may be performed by a dynamic image data-based object state prediction device 100 (hereinafter referred to as an “object state prediction device”).

The object state prediction device 100 may obtain early dynamic image data 10. The early dynamic image data 10 may correspond to an early section up to a preset time point after a medicine is injected into an object through a tracer. The early dynamic image data 10 may include a plurality of image frames, each of which may be acquired in a 1-minute or 2-minute period, but the present disclosure is not limited thereto.

Here, the preset time point may be a time point within a predetermined range based on the peak of the radiation dose included in the blood flow of the object. Moreover, the early section may vary depending on the type of tracer and the temperamental factors of the diagnostic object.

The object state prediction device 100 may input the acquired early dynamic image data 10 into a first prediction model EM1 and a second prediction model EM2 (S11 and S12).

In this case, early dynamic image data corresponding to a first section in the early dynamic image data 10 may be input to the first prediction model EM1, and early dynamic image data corresponding to a second section in the early dynamic image data 10 may be input to the second prediction model EM2. However, according to an embodiment, the object state prediction device 100 may input the same early dynamic image data 10 to the first prediction model EM1 and the second prediction model EM2.

The first prediction model EM1 may predict true early image data 20 based on the input early dynamic image data (S21). The true early image data may be dynamic image data corresponding to a preset short period after medicine injection or image data at a specific time point.

Furthermore, the second prediction model EM2 may predict delay image data 30 based on the input early dynamic image data (S22). The delay image data may be dynamic image data corresponding to a reference time point after an initial period or image data at a specific time point.

In addition, the early dynamic image data corresponding to the first section, the early dynamic image data corresponding to the second section, the true early image data, and the delay image data may all be positron emission tomography (PET) image data, but the present disclosure is not limited thereto.

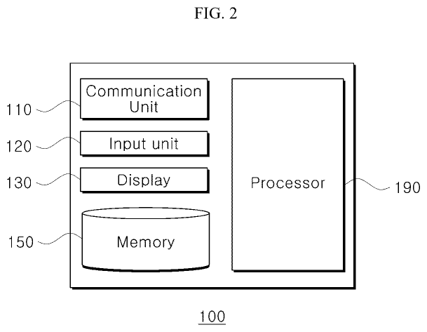

FIG. 2 is a block diagram showing a configuration of the object state prediction device 100, according to an embodiment of the present disclosure.

Referring to FIG. 2, the object state prediction device 100 may include a medical image capturing device or may utilize image data captured from a medical image capturing device, and may include a communication unit 110, an input unit 120, a display 130, a memory 150, and the at least one processor 190. The components of the object state prediction device 100 shown in FIG. 2 are not essential in implementing the object state prediction device 100. The object state prediction device 100 described herein may have more or fewer components than those listed above.

Among the components, the communication unit 110 may include one or more components that enable communication with various devices equipped with a communication device, and may include, for example, at least one of a satellite device, a wired communication device, a cellular-based wireless communication device, an IEEE 802.11-based (e.g., it may be called “WiFi”) wireless communication device, a short-range communication-based communication device (e.g., it may be Bluetooth, Bluetooth Low Energy, UWB, Zigbee, but is not limited thereto), and a location information module.

The input unit 120 may be used to input image information (or signal), audio information (or signal), data, or information entered from a user, and may include, but is not limited to, at least one camera, a touch input device provided on a touch screen, and/or at least one microphone. A touch input for the touch screen collected from the input unit 120, voice data, and/or image data may be analyzed and then processed as a user's control command. The input unit 120 may include a device (an inputter) for various inputs.

The camera may process an image frame such as a still image or a moving image, which is obtained by an image sensor in a shooting mode. The processed image frame may be displayed on the display 130 (or the screen of the object state prediction device 100 according to an embodiment of the present disclosure) or stored in the memory 150.

The microphone processes external acoustic signals into electrical speech data. The processed speech data may be used in various ways depending on a function (or a running application) being performed by the apparatus. In the meantime, various noise cancellation algorithms for canceling noise generated in a process of receiving an external sound signal may be implemented in a microphone.

The output unit may be used to generate an output related to visual, auditory or tactile sensations, and may include at least one of the display 130, at least one speaker, a haptic module, and an optical output device. The display 130 may have a mutual layer structure with a touch input device or may be integrally formed with the touch input device, thereby implementing a touch screen. These touch screens may perform an output function and/or an input function. The output device may include an output (an outputter) for various outputs.

The memory 150 may store at least one instruction that causes the object state prediction device 100 to perform various functions. The memory 150 may store data (e.g., music files, still images, videos, etc.) for content expression. The memory 150 may store at least one application program (or application) that causes operations performed by various embodiments of the present disclosure driven by the object state prediction device 100 to be performed, pieces of data for the operation of the object state prediction device 100, and commands for the operation of the object state prediction device 100. At least part of the application programs may be downloaded from an external server through wireless communication. For example, the object state prediction device 100 may download an application and may store the application in the memory 150. The object state prediction device 100 may perform operations performed by various embodiments of the present disclosure by executing an application. Alternatively, the object state prediction device 100 may temporarily download data (for example) that causes the device to perform operations performed by various embodiments of the present disclosure from a server and may store the data in the memory 150.

The memory 150 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc.

It may be understood by one skilled in the art that the memory 150 may also mean, for example, a cache memory for connection with the processor 190, and/or a cache memory and/or registers included in the processor 190. Furthermore, the memory 150 may be separate from the object state prediction device 100, but may be a database connected by wire or wirelessly, and may be implemented as a database system.

The processor 190 may include one or more processors, each of which may include at least one core. The processor 190 may execute an instruction stored in the memory 150. The processor 190 may be implemented with a memory that stores data regarding an algorithm for controlling operations of components within the object state prediction device 100, or a program for implementing the algorithm, and the at least one processor that performs the above-described operation by using the data stored in the memory. At this time, the memory and the processor may be implemented as separate chips. Alternatively, the memory and the processor may be implemented as a single chip.

In an embodiment, the object state prediction device 100 may provide various UIs in the form of web services based on a platform, such as, but not limited to, a website or a web application. Moreover, the corresponding platform may be provided in the form of a PC application, a mobile application, etc., but an embodiment is not limited thereto. In this case, various user terminals may use various UIs provided by the object state prediction device 100 based on the platform.

The predefined operating rule or the artificial intelligence model is created through learning. Here, being created through learning means creating the predefined operating rule or the artificial intelligence model configured to perform desired features (or purposes) as a basic artificial intelligence model is learned by using pieces of learning data by a learning algorithm. This learning may be performed by a device itself, on which the artificial intelligence according to an embodiment of the present disclosure is performed, or may be performed through a separate server and/or system. For example, the learning algorithm may include supervised learning, unsupervised learning, semi-supervised learning, or reinforcement learning, but may not be limited to the above example.

An artificial intelligence model may be composed of a plurality of neural network layers. The plurality of neural network layers respectively have a plurality of weight values, and each of the plurality of neural network layers performs neural network calculation through calculations between the calculation result of the previous layer and the plurality of weight values. The plurality of weight values of the plurality of neural network layers may be optimized by the learning result of the artificial intelligence model. For example, during a learning process, the plurality of weight values may be updated such that a loss value or a cost value obtained from the artificial intelligence model is reduced or minimized. The artificial neural network may include a deep neural network (DNN). The artificial neural network may be, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the above-described example.

According to an embodiment of the present disclosure, a processor may implement artificial intelligence. The artificial intelligence may refer to an artificial neural network-based machine learning method that allows a machine to perform learning by simulating human biological neurons. The methodology of artificial intelligence may be classified as supervised learning, in which a solution (output data) to a problem (input data) is determined by providing input data and output data together as training data depending on a learning method, unsupervised learning, in which only input data is provided without output data, and thus the solution (output data) to the problem (input data) is not determined, and reinforcement learning, in which a reward is given from an external environment whenever an action is taken in a current state, and thus learning progresses to maximize this reward. Moreover, the methodology of artificial intelligence may also be categorized depending on architecture, which is the structure of the learning model. The architecture of deep learning technology widely used may be categorized into convolutional neural networks (CNN), recurrent neural networks (RNN), transformers, and generative adversarial networks (GAN).

Each of the present device and the system may include an artificial intelligence model. The artificial intelligence model may be a single artificial intelligence model or may be implemented as a plurality of artificial intelligence models. The artificial intelligence model may be composed of neural networks (or artificial neural networks) and may include a statistical learning algorithm that mimics biological neurons in machine learning and cognitive science. The neural network may refer to a model as a whole having the ability to solve problems as artificial neurons (nodes), which form a network by connecting synapses, changes the strength of their synaptic connections through learning. Neurons in the neural network may include the combination of weight values or biases. The neural network may include one or more layers consisting of one or more neurons or nodes. For example, the present device may include an input layer, a hidden layer, and an output layer. The neural network constituting the present device may infer the result (output) to be predicted from an arbitrary input by changing a weight value of a neuron through learning.

The processor may create a neural network, may train or learn a neural network, or may perform operations based on received input data, and then may generate an information signal or may retrain the neural network based on the performed results. Models of a neural network may include various types of models such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) Network, and a classification network, but is not limited thereto. The processor may include one or more processors for performing computations according to the models of the neural network. For example, the neural network may include a deep neural network.

It will be understood by those skilled in the art that a neural network may include any neural network, but is not limited to a convolutional neural network (CNN), a recurrent neural network (RNN), a perceptron, a multilayer perceptron, a feed forward (FF), a radial basis network (RBF), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN).

At least one component may be added or deleted to correspond to the performance of the components illustrated in FIG. 2. Furthermore, it will be easily understood by those skilled in the art that mutual locations of the components may be changed to correspond to the performance or structure of the system.

In the meantime, each component shown in FIG. 2 refers to software components and/or hardware components such as field programmable gate array (FPGA) and application specific integrated circuit (ASIC).

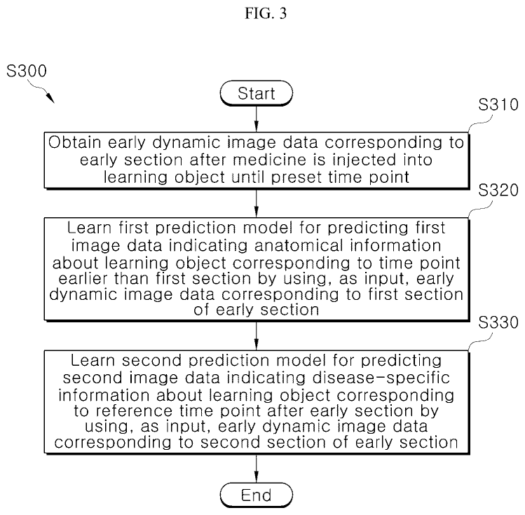

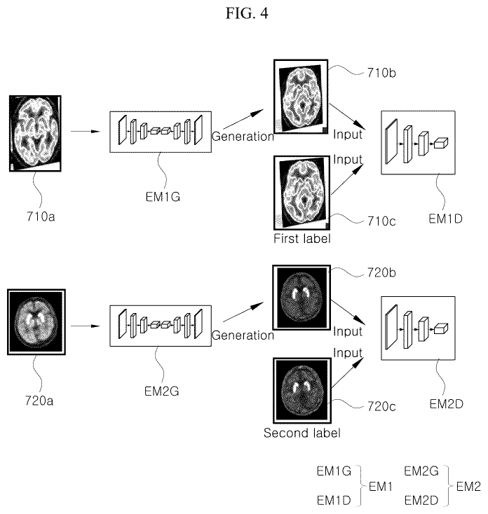

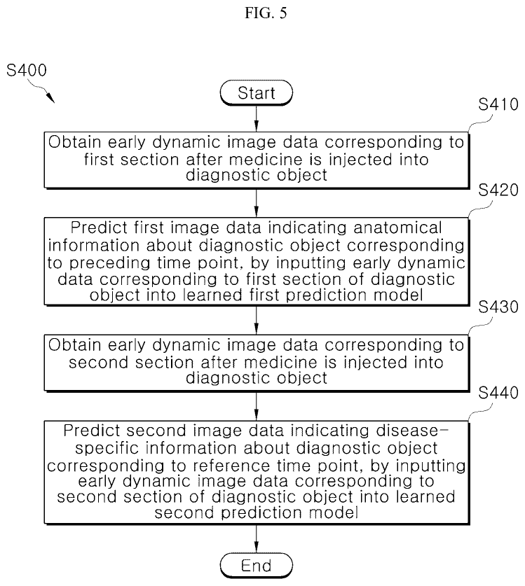

FIG. 3 is a sequence diagram illustrating an object state prediction method S300 (including operations S310 to S330) based on dynamic image data, according to an embodiment of the present disclosure. FIG. 4 is a diagram for describing a process of learning the first prediction model EM1 and the second prediction model EM2 based on early dynamic image data, according to an embodiment of the present disclosure. A part required while an embodiment of FIG. 3 is described will refer to FIG. 4 together.

In operation S310, the processor 190 may obtain early dynamic image data corresponding to an early section after a medicine is injected into a learning object until a preset time point.

Here, the medicine may be a tracer. The tracer may have various types depending on a target tissue or an organ. For example, different types of tracers may be applied when the location or size of a tumor is identified, the functional activity of a brain is investigated, or cardiac blood flow and metabolic activity is diagnosed.

Moreover, the preset time point may be a time point within the preset range at a point at which the radiation dose of a site other than the target tissue or organ peaks, and may be used as a default value according to an embodiment.

In operation S320, the processor 190 may learn the first prediction model EM1 for predicting first image data indicating anatomical information (or blood flow information) about the learning object, corresponding to a time point earlier than a first section by using, as an input, early dynamic image data corresponding to the first section of the early section. Here, the first image data may be true early image data.

Referring to FIG. 4, the first prediction model EM1 may include a generation model EM1G and a determination model EM1D.

When inputting early dynamic image data 710a corresponding to the first section into the generation model EM1G of the first prediction model EM1, the processor 190 may predict (generate) middle true early image data 710b.

The processor 190 may compare and verify a first label 710c and the middle true early image data 710B generated through the generation model EM1G, by inputting the generated middle true early image data 710B and the label image data 710c (the first label) of the true early image data into the determination model EM1D. The processor 190 may repeatedly perform generation and determination until the first prediction model EM1 satisfies a preset condition (e.g., similarity of 95% or higher).

The first prediction model EM1 may be generated based on a GAN model and a conditional GAN model that additionally utilizes condition information (e.g., label) may be applied, but the present disclosure is not limited thereto.

In operation S330, the processor 190 may learn the second prediction model EM2 for predicting second image data indicating disease-specific information about the learning object, corresponding to a reference time point after the early section by using, as an input, early dynamic image data corresponding to a second section of the early section. Here, the second image data may be delay image data.

Referring to FIG. 4, when inputting early dynamic image data 720a corresponding to the second section into a generation model EM2G of the second prediction model EM2, the processor 190 may predict middle delay image data 720b.

The processor 190 may compare and verify a second label 720c and the middle delay image data 720b generated through the generation model EM2G, by inputting the generated middle delay image data 720b and label image data 720c (the second label) of the delay image data into a determination model EM2D. The processor 190 may repeatedly perform generation and determination until the second prediction model EM2 satisfies a preset condition (e.g., similarity of 95% or higher).

The second prediction model EM2 may be generated based on a GAN model and a conditional GAN model that additionally utilizes condition information (e.g., label) may be applied, but the present disclosure is not limited thereto.

In an embodiment, the amount of medicine injected into a learning object may be an amount reduced from the reference amount. For example, the amount reduced from the reference amount may be half of the reference amount, but the present disclosure is not limited thereto.

The first prediction model EM1 may be learned based on the first label image data corresponding to the true early image data. The second prediction model EM2 may be learned based on the second label image data corresponding to the delay image data. In this case, the first label image data and the second label image data may be label image data processed according to the medicine injection amount of the learning object. When the amount of medicine injected into the learning object is half of the reference amount, the processor 190 needs to use the first and second label image data as label image data corresponding to half of the reference amount.

In an embodiment, the processor 190 may directly obtain first label image data and second label image data, which are associated with a case where the medicine injection amount is less than the reference amount, through clinical trials and may use them for learning.

However, even when an amount of medicine smaller than the reference amount is injected, government approval is required, and thus it may be difficult to obtain the data. Accordingly, when generating the first label image data and the second label image data, which are associated with the case where the medicine injection amount is equal to the reference amount, the processor 190 may reduce the frame generation time to generate the first label image data and the second label image data corresponding to a case where the medicine injection amount is less than the reference amount.

For example, when generating five pieces of image data frames every two minutes, the processor 190 may generate image data every one minute, may acquire frames every two minutes (not using image data originally generated every two minutes), and may use the acquired image data as the first label image data and the second image data. That is, the processor 190 may generate the first label image data and the second label image data corresponding to the injection amount that is half of the reference amount. In other words, the processor 190 may process images corresponding to a case where a medicine injection amount is reduced, to be generated by using list mode data, or may generate image frames at 1-minute intervals, not generate image frames at 2-minute intervals by applying an approximate method.

According to the implementation example, this may be performed by changing the order between operations S320 and S330.

FIG. 5 is a sequence diagram showing a method S400 for predicting first image data indicating anatomical information of a diagnosis subject and second image data indicating disease-specific information, according to an embodiment of the present disclosure.

In operation S410, the processor 190 may obtain early dynamic image data corresponding to a first section after a medicine is injected into a diagnostic object.

In operation S420, the processor 190 may predict first image data indicating anatomical information (or blood flow information) about the diagnostic object corresponding to the preceding time point, by inputting early dynamic image data corresponding to a first section of the diagnostic object into the learned first prediction model EM1.

Here, the first image data may be true early image data, and the first section may be a period between 10 minutes and 30 minutes after medicine injection, but an embodiment is not limited thereto. The preceding time point is a time point corresponding to the true early image data, and may be within a preset range (e.g., 1 minute) after medicine injection, but an embodiment is not limited thereto.

In operation S430, the processor 190 may obtain early dynamic image data corresponding to a second section after the medicine is injected into the diagnostic object.

The second section may be a period between 10 minutes and 20 minutes after medicine injection, but the present disclosure is not limited thereto.

In operation S440, the processor 190 may predict second image data indicating disease-specific information about the diagnostic object corresponding to the reference time point, by inputting early dynamic image data corresponding to a second section of the diagnostic object into the learned second prediction model EM2.

In an embodiment, the processor 190 may inject a medicine reduced by a preset amount into the diagnostic object, and may obtain and process early dynamic image data corresponding to the first section and early dynamic image data corresponding to the second section based thereon.

Here, the medicine may be injected by an amount reduced from the reference amount by a preset amount to reduce an injection radiation dose.

The processor 190 may process early dynamic image data corresponding to the first section and early dynamic image data corresponding to the second section before inputting them into the prediction models EM1 and EM2. Because the injection radiation dose is less than the reference amount, the processor 190 processes the early dynamic image data as early dynamic image data corresponding to the case where the reference amount is injected.

The processor 190 may process the movement path of the medicine within a human body more vividly and clearly from the early dynamic image data corresponding to the first section, and may process each pixel value into each pixel value corresponding to a case, where the reference amount is injected, by using a smoothing filter (e.g., a Gaussian filter). That is, the processor 190 may process each pixel value by using a smoothing technique (e.g., a spatial smoothing method).

In an embodiment, when inputting early dynamic image data corresponding to the first section and the second section, which correspond to a case where a small amount of medicine is injected, into a processing model, the processor 190 may also output and use the early dynamic image data corresponding to the first section and the early dynamic image data corresponding to the second section, which correspond to a case where the reference amount is been injected, through the processing model.

After obtaining the processed early dynamic image data corresponding to the first section and the processed early dynamic image data corresponding to the second section, the processor 190 may input the processed early dynamic image data corresponding to the first section into the first prediction model EM1 to predict the first image data indicating anatomical information corresponding thereto, and may input the processed early dynamic image data corresponding to the second section into the second prediction model EM2 to predict the second image data indicating disease-specific information corresponding thereto.

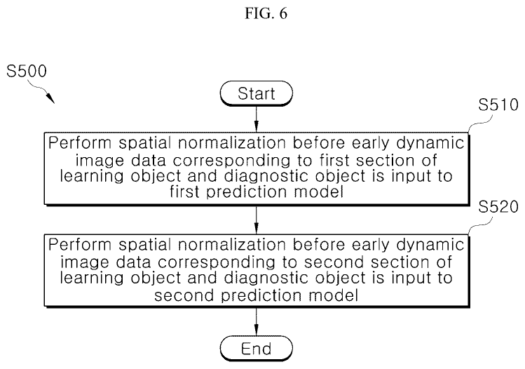

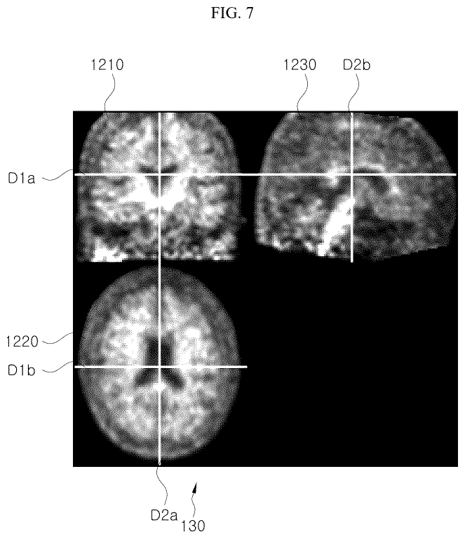

FIG. 6 is a sequence diagram showing a preprocessing process S500 (including operations S510 and S520) for performing spatial normalization, according to an embodiment of the present disclosure. FIG. 7 is a diagram for describing spatial normalization performed in a preprocessing step before learning an artificial neural network model, according to an embodiment of the present disclosure.

In operation S510, the processor 190 may perform spatial normalization such that machine learning through a smaller number of learning object material is possible, before early dynamic image data corresponding to a first section of a learning object and a diagnostic object is input to the first prediction model EM1.

In operation S520, the processor 190 may perform spatial normalization before early dynamic image data corresponding to a second section of a learning object and a diagnostic object is input to the second prediction model EM2.

Referring to FIG. 7, before inputting the early dynamic image data corresponding to the first section of the learning object and the diagnostic object to the first prediction model EM1, or inputting the early dynamic image data corresponding to the second section of the learning object and the diagnostic object to the second prediction model EM2, the processor 190 may perform spatial normalization on a first image 1210, a second image 1220, and a third image 1230 based on a reference site, by performing spatial normalization on the early dynamic image data.

For example, the processor 190 may place, in a space, the first image 1210 based on a reference center by using a horizontal line D1a and a vertical line D2a, may place, in the space, the second image 1220 based on the reference center by using the horizontal line D1a and the vertical line D2a, and may place, in the space, the third image 1230 based on the reference center by using the horizontal line D1a and the vertical line D2a. Accordingly, learning efficiency may be improved and diagnosis may be effective.

The processor 190 may set an acquisition time section of early dynamic image data corresponding to the first section and/or the second section in the early section of the learning object and the diagnostic object to consider the operability of equipment and the predictability of diagnosis subjects.

The processor 190 may identify an early section when a medicine is injected into a diagnostic object, and may obtain early dynamic image data corresponding to the first section and the second section based on the identified early section.

The processor 190 may set the time required to obtain the early dynamic image data corresponding to the first section and the second section depending on individual characteristics, but the present disclosure is not limited thereto.

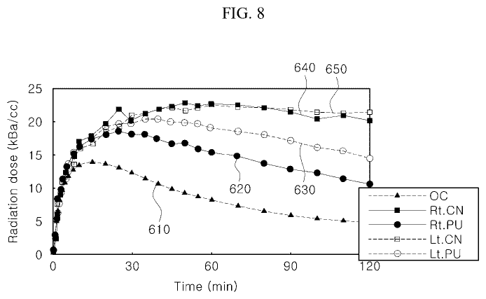

FIG. 8 is a diagram showing changes in radiation dose over time after medicine injection, according to an embodiment of the present disclosure.

Referring to FIG. 8, when FP-CIT is used as a drug (i.e., a tracer), diagnosis may be difficult due to high radiation doses in entire regions of a brain due to blood flow effects in an initial time range in which the drug is administered. However, as time elapses, a decrease in the drug in the blood may be identified as the drug is excreted in the urine or metabolized by the liver, thereby causing the concentration in the plasma to decrease. Furthermore, the drug is released from brain tissues into a blood to balance the decrease in drug levels in the blood. The processor 190 may obtain early dynamic image data from an early section where a radiation dose within the brain tissues changes.

Afterwards, because the FP-CIT has a strong binding affinity with dopamine and is effective in determining Parkinson's disease, the concentration in other regions 610 to 630 decreases due to reduced blood flow, but target sites 640 and 650 maintains a high concentration when the FP-CIT binds to the dopamine neurotransmitter being a target site. The processor 190 may determine an early section of the early dynamic image data based on a point where the dose concentration of another region (e.g., the reference regions 610 to 630) peaks.

The processor 190 may obtain delay image data at a time point where the blood flow effect is reduced after a reference time elapses after the drug is injected. The delay image data may be image data previously used by medical staff to diagnose the size and shape of the target site.

In an embodiment, when the medicine used to capture image data is a tracer that binds to a specific target region, the early section for acquiring the early dynamic image data may be determined based on various criteria. For example, the early section may be set to a time range after a time point at which the influence of blood flow in the reference region begins to decrease. In general, the reference region may be a specific site of a brain that does not differ between diseases because there is no substance that the tracer binds to. The reference region varies depending on the type and characteristics of the tracer. For example, when the tracer is FP-CIT, the reference region corresponds to a site with low dopamine neurotransmitter, such as the cerebellum or occipital cortex (OC). Moreover, for another example, the early section may be set to the periphery of a time point, where a dose ratio difference value between the target region and the reference region is greater than or equal to a certain value or a ratio difference value reaches a maximum.

When the medicine used to obtain image data is a tracer (e.g., Fluorodeoxyglucose (referred to as “FDG”) PET) whose binding to the target region increases over time, the dose to each brain region increases without a peak point. Accordingly, the processor 190 may obtain early dynamic image data in the early section, where a radiation dose difference is not significant, and may obtain delay image data at a reference time point, where the radiation dose difference ratio between regions increases.

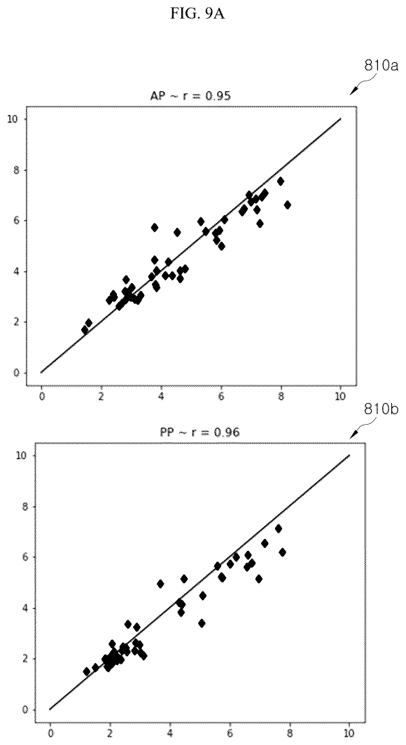

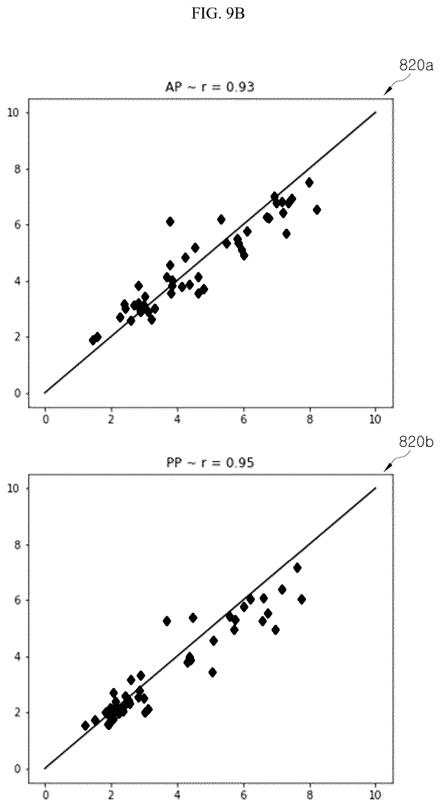

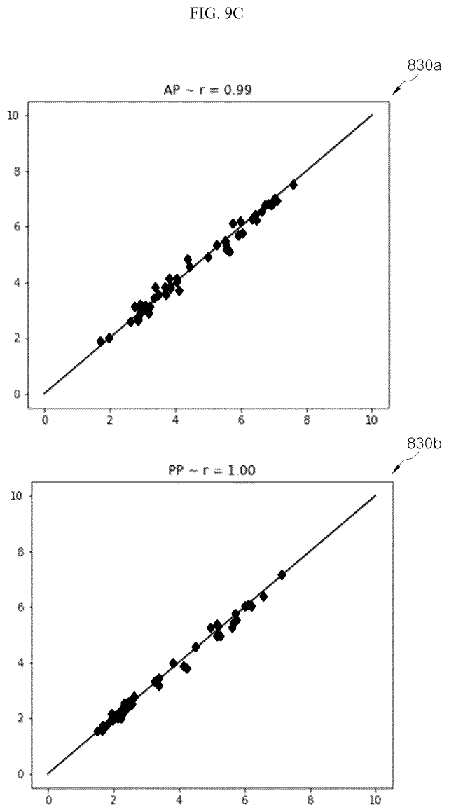

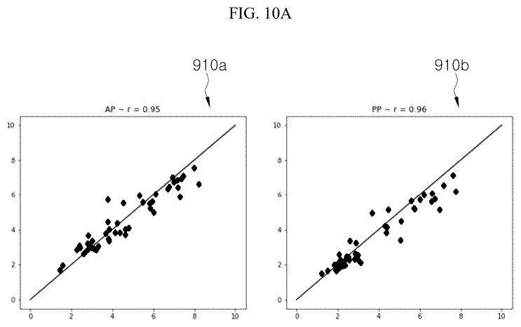

FIG. 9A is a graph for comparing label image data with delay image data generated by using a reference dose, according to an embodiment of the present disclosure. FIG. 9B is a graph for comparing label image data with delay image data generated by using a low dose, according to an embodiment of the present disclosure. FIG. 9C is a graph for comparing delay image data generated by using a reference dose with delay image data generated by using a low dose according to an embodiment of the present disclosure.

Referring to FIG. 9A, as comparing Dopamine Transporter (DAT) uptake between label image data and delay image data generated by using a reference dose, a correlation in Anterior Putamen (AP) (the front part of the basal ganglia) may be calculated as 0.95, and a correlation in Posterior Putamen (PP) (the back part of the basal ganglia) may be calculated as 0.96.

Referring to FIG. 9B, as comparing DAT uptake between label image data and delay image data generated by using a low dose (half of a reference dose), a correlation in AP may be calculated as 0.93, and a correlation in PP (the back part of the basal ganglia) may be calculated as 0.95.

Referring to FIG. 9C, as comparing DAT uptake between delay image data generated by using the reference dose and delay image data generated by using the low dose, a correlation in AP may be calculated as 0.99, and a correlation in PP between delay image data generated by using the reference dose and delay image data generated by using the low dose may be calculated as 1.00.

Referring to FIGS. 9A to 9C, an image is obtained in a state that is less attenuated by the half-life being characteristic of nuclear medicine molecular images, and thus delayed uptake images of the same quality may be predicted even with low-dose image obtained by reducing an isotope injection amount.

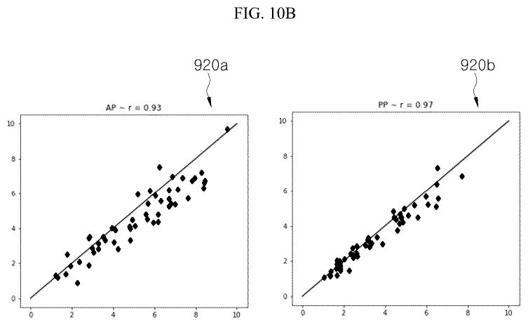

FIG. 10A illustrates analog PET data used in training and testing steps for each of AP and PP, according to an embodiment of the present disclosure. FIG. 10B illustrates digital PET data used in the test step for each of the AP and the PP, according to an embodiment of the present disclosure.

Referring to FIG. 10A, with respect to analog PET data, it may be identified that the test set results indicate that a correlation in AP is 0.95 and a correlation in PP is 0.96.

Referring to FIG. 10B, with respect to digital PET data, it may be identified that independent test sets indicate that the correlation in AP is 0.93 and the correlation in PP is 0.97.

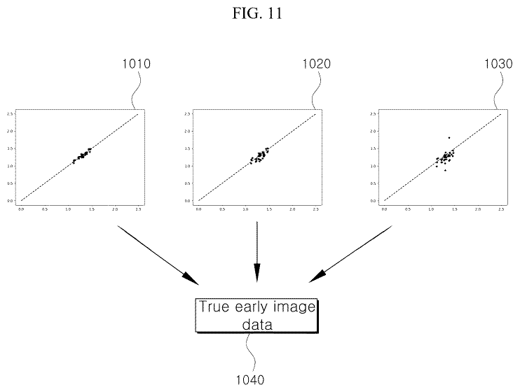

FIG. 11 is a diagram for describing a method for obtaining true early image data, according to an embodiment of the present disclosure.

It may be identified that a correlation between true early image data and dynamic image data, which is obtained when 10 minutes elapses after medication injection within an early section, is 0.94 (1010); it may be identified that a correlation between the true early image data and the dynamic image data, which is obtained when 20 minutes elapses after medication injection, is 0.77 (1020); and it may be identified that a correlation between the true early image data and the dynamic image data, which is obtained when 30 minutes elapses after medication injection, is 0.53 (1030). In this way, the highest correlation may be identified in the case of dynamic image data, which is obtained when 10 minutes elapses after medicine injection and which is closest in time to the true early image data.

The processor 190 may predict true early image data 1040 based on dynamic image data obtained when 10 to 30 minutes elapses after medicine injection. That is, the true early image data 1040 may be effectively predicted by using early dynamic image data (e.g., the first section of the early section).

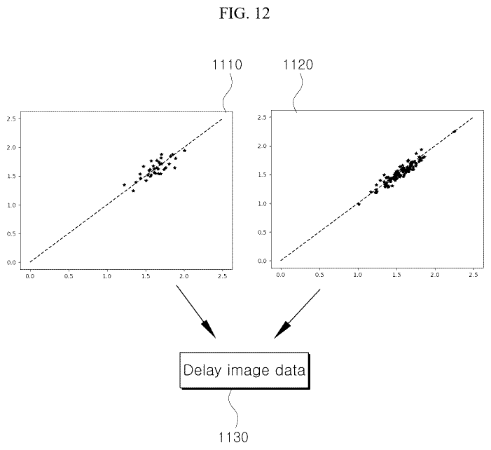

FIG. 12 is a diagram for describing a method for obtaining delay image data, according to an embodiment of the present disclosure.

It may be identified that a correlation between delay image data and dynamic image data, which is obtained when 10 minutes elapses after medication injection within an early section, is 0.80 (1110), and it may be identified that a correlation between the delay image data and the dynamic image data, which is obtained when 20 minutes elapses after medication injection, is 0.94 (1120). In this way, the highest correlation may be identified in the case of dynamic image data, which is obtained when 20 minutes elapses after medicine injection and which is closest in time to the delay image data.

The processor 190 may predict delay image data 1130 based on dynamic image data obtained when 10 to 20 minutes elapses after medicine injection. That is, the delay image data 1130 may be effectively predicted by using early dynamic image data (e.g., the second section of the early section).

Meanwhile, the quality of dynamic image data may be set according to the number of frames thus required. That is, the maximum amount of radiation emitted externally may be restricted when the same amount of drug is used, thereby limiting the amount of signal used to generate an image frame. Accordingly, a computing device may differently set the image quality of each image frame depending on the number of image frames, based on the same amount of signal.

For example, increasing the number of image frames reduces the length of time used to generate each image frame, and thus the amount of signal available to generate one image frame is reduced, and the computing device generates each image frame at a lower quality.

In an embodiment, the computing device may perform learning by matching changes in each pixel in the dynamic image data over time with changes in each pixel in the initial image data and delay image data.

In other words, in the combination (i.e., the combination of early dynamic image data, dynamic image data, and delay image data) of image data for a specific patient, the computing device predicts early dynamic image data and delay image data for each point (i.e., a pixel) of a body tissue (e.g., a brain tissue) from the dynamic image data, builds a dataset for each image data combination by matching anatomical information in the early dynamic image data with disease-specific information in the delay image data, and builds a diagnostic image prediction model by learning dataset for a plurality of patient for each point (i.e., a pixel).

In an embodiment, the diagnostic image prediction model is built by using a deep neural network (DNN). In other words, the diagnostic image prediction model learns dynamic image data for one or more patients by applying a deep learning algorithm.

A processor may generate image data by normalizing the brightness of dynamic image data based on a maximum value or an average value.

In an embodiment, when a medicine used to capture image data is a tracer (e.g., Fluorodeoxyglucose (FDG) PET), of which the amount continuously binding to the target region increases over time, the brightness ratio between a reference region and a target region does not change with the drug dose due to the characteristic of PET tracers that are uptaken in proportion to the drug dose. Accordingly, the computing device normalizes the dynamic image data based on the maximum value or the average value of brightness and then generates image data.

In the meantime, in the present disclosure, the operation is described by using PET. However, the above-described operation may be applied not only to PET but also to MR and may be applied to medical images in general.

However, in the case of MR, disease-specific information may be derived through the early dynamic image data, and anatomical information may be derived through the delay image data.

When the medicine used to capture image data is a tracer that binds to a specific target region (e.g., FP-CIT is used as the tracer), a tracer model parameters remain constant even when the input and output are linearly increased and decreased simultaneously in a linear tracer kinetic model using the reference region as an input and using the target region as an output, and thus the computing device may generate new delay image data by normalizing (count normalization or intensity normalization) the brightness of learning-specific early dynamic image data and the diagnostic early dynamic image data based on the maximum value or the average value.

In other words, when the medicine used to capture image data is a tracer that binds to a specific target region, the learning-specific dynamic image data may be formed as image data acquired from a point in time when a dose ratio difference value between the target region and the reference region is greater than or equal to a specific value or reaches the maximum value, to a point in time when the influence of blood flow is eliminated.

Moreover, the learning-specific dynamic image data may be formed as image data acquired from a point in time when a dose ratio difference value between the target region and the reference region is greater than or equal to a specific value or a dose change amount of the reference region due to blood flow decreases, to a point in time when a dose ratio difference value between the target region and the reference region reaches the maximum value.

The dose ratio value may mean a ratio between the radiation dose at an initial time point and the radiation dose at a specific measurement time point.

Accordingly, the dose ratio difference value may mean a difference value between a dose ratio of the target region and a dose ratio of the reference region.

In detail, the processor may obtain information about the dose of each of the target image and the reference image.

Furthermore, the processor may obtain a dose corresponding to the target image, and a dose corresponding to a reference image, may calculate a ratio value of each dose, and may calculate the difference value between the dose ratios.

Besides, the processor may determine a time point at which the dose variation of the reference region decreases, and a time point at which the dose ratio difference is maximized.

In general, when making a diagnosis based on delay image data, medical staff administers a large amount of medicine at an initial time point so as to make a diagnosis based on delay image data acquired with a sufficient radiation dose even after the radioisotope decreases according to its physical half-life and the decrease due to elimination from the body, such as urination/defecation (i.e., physiologic decay). In this case, the radiation dose provided to the patient's body may increase.

Accordingly, according to an embodiment of the present disclosure, the amount of radioactive material injected into the patient may be reduced to reduce the radiation dose delivered to a patient's body, by obtaining diagnostic early dynamic image data after only enough tracers are inserted to obtain sufficient radiation dose when the diagnostic early dynamic image data is obtained, and then inserting the diagnostic early dynamic image data into a prediction model to obtain early dynamic image data and final diagnostic delay image data.

The predetermined reference time may be determined based on the type of tracer.

In the meantime, the processor may generate second learning data for predicting anatomical and disease-specific information from the early dynamic image data and the delay image data based on a second diagnostic image prediction model using the early dynamic image data and the delay image data.

In summary, a first diagnostic prediction model may predict early dynamic image data and delay image data from dynamic image data, and may learn and predict anatomical and disease-specific information corresponding thereto. On the other hand, the second diagnostic prediction model may perform learning based on the early dynamic image data and the delay image data corresponding to the dynamic image data, and may then predict anatomical and disease-specific information in an integrated manner when the diagnostic early dynamic image data or the delay image data is input.

Meanwhile, the processor may determine the state of the object based on the anatomical information and the disease-specific information.

In detail, the processor may derive information about whether an object corresponds to Parkinson's disease or dementia, by using the disease-specific information and the anatomical information of the object derived in the above-described method.

In particular, when a tracer remains in a dopamine neurotransmitter of the object, the object may be determined to be in a state of Parkinson's disease.

Moreover, when the tracer of amyloid remains, the object may be determined to be in a dementia state.

The processor may determine a diagnostic image prediction model having a higher accuracy between the accuracy of the first diagnostic image prediction model and the accuracy of the second diagnostic image prediction model.

For example, when making a diagnosis by using early dynamic image data, a diagnostician may calculate the accuracy of the first diagnostic prediction model for Parkinson's disease, and the accuracy of the second diagnostic prediction model for Parkinson's disease. In this case, the processor may determine the state of the object by selecting a diagnostic prediction model with high accuracy among the diagnostic prediction models.

Under detailed examination conditions such as appropriate dynamic image acquisition time point, the early dynamic image data-based diagnostic image generation method according to an embodiment of the present disclosure may be implemented by a program (or an application) and may be stored in a medium such that the program is executed in combination with a computer being hardware.

The early dynamic image data-based diagnostic image generation method according to an embodiment of the present disclosure may be implemented by a program (or an application) and may be stored in a medium such that the program is executed in combination with a computer being hardware.

The above-described program may include a code encoded by using a computer language such as C, C++, JAVA, a machine language, or the like, which a processor (CPU) of the computer may read through the device interface of the computer, such that the computer reads the program and performs the methods implemented with the program. The code may include a functional code related to a function that defines necessary functions executing the method, and the functions may include an execution procedure related control code necessary for the processor of the computer to execute the functions in its procedures. Furthermore, the code may further include a memory reference related code on which location (address) of an internal or external memory of the computer should be referenced by the media or additional information necessary for the processor of the computer to execute the functions. Further, when the processor of the computer is required to perform communication with another computer or a server in a remote site to allow the processor of the computer to execute the functions, the code may further include a communication related code on how the processor of the computer executes communication with another computer or the server or which information or medium should be transmitted/received during communication by using a communication module of the computer.

The stored medium refers not to a medium, such as a register, a cache, or a memory, which stores data for a short time but to a medium that stores data semi-permanently and is read by a device. Specifically, for example, the stored media include, but are not limited to, ROM, RAM, CD-ROM, magnetic tape, floppy disk, optical data storage device, and the like. That is, the program may be stored in various recording media on various servers, which the computer may access, or in various recording media on the computer of the user. Further, the media may be distributed in computer systems connected over a network such that codes readable by the computer are stored in a distributed manner.

The uptake time, which is characteristic of the nuclear medicine molecule image, is several hours after the injection of the medicine, waiting for the distribution of the image tracer in a body. According to various embodiments of the present disclosure, after medicine injection, early blood flow image data and delayed intake image data may be generated simultaneously with only early dynamic image data, and the waiting time for diagnosis may be dramatically reduced, thereby providing great user convenience.

Although an embodiment of the present disclosure are described with reference to the accompanying drawings, it will be understood by those skilled in the art to which the present disclosure pertains that the present disclosure may be carried out in other detailed forms without changing the scope and spirit or the essential features of the present disclosure. Therefore, the embodiments described above are provided by way of example in all aspects, and should be construed not to be restrictive.

What is claimed is:

1. A method for predicting object state based on dynamic image data, performed by at least one processor, the method comprising:
obtaining early dynamic image data corresponding to an early section after a medicine is injected into a learning object until a preset time point;
learning a first prediction model for predicting first image data indicating anatomical information or blood flow information about the learning object corresponding to a time point earlier than a first section by using, as an input, early dynamic image data corresponding to the first section of the early section; and
learning a second prediction model for predicting second image data indicating disease-specific information about the learning object corresponding to a reference time point after the early section by using, as an input, early dynamic image data corresponding to a second section of the early section.

2. The method of claim 1, further comprising:
obtaining early dynamic image data corresponding to the first section after the medicine is injected into a diagnostic object; and
predicting first image data indicating anatomical information or blood flow information about the diagnostic object corresponding to the earlier time point, by inputting the early dynamic image data corresponding to the first section of the diagnostic object into the learned first prediction model.

3. The method of claim 2, further comprising:
obtaining early dynamic image data corresponding to the second section after the medicine is injected into the diagnostic object; and
predicting second image data indicating disease-specific information about the diagnostic object corresponding to the reference time point, by inputting the early dynamic image data corresponding to the second section of the diagnostic object into the learned second prediction model.

4. The method of claim 3, wherein the processor is configured to:
when the medicine reduced by a preset amount is injected into the diagnostic object, obtain and process early dynamic image data corresponding to the first section and early dynamic image data corresponding to the second section based on the injection to the diagnostic object, and obtain the processed early dynamic image data corresponding to the first section and the processed early dynamic image data corresponding to the second section; and
input the processed early dynamic image data corresponding to the first section into the first prediction model, and input the processed early dynamic image data corresponding to the second section into the second prediction model.

5. The method of claim 3, wherein an amount of the medicine injected into the learning object is an amount reduced from a reference amount,
wherein the first prediction model is learned based on first label image data corresponding to the first image data, and the second prediction model is learned based on second label image data corresponding to the second image data, and
wherein each of the first label image data and the second label image data is label image data processed according to an medicine injection amount of the learning object.

6. The method of claim 3, further comprising:
performing spatial normalization before the early dynamic image data corresponding to the first section of the learning object and the diagnostic object is input to the first prediction model; and
performing spatial normalization before the early dynamic image data corresponding to the second section of the learning object and the diagnostic object is input to the second prediction model.

7. The method of claim 3, wherein the processor is configured to:
set an acquisition time section of the early dynamic image data corresponding to the first section in the early section of the learning object and the diagnostic object; and
set an acquisition time section of the early dynamic image data corresponding to the second section in the early section of the learning object and the diagnostic object.

8. The method of claim 3, wherein the processor is configured to:
identify the early section when the medicine is injected into the diagnostic object; and
obtain the early dynamic image data corresponding to the first section and the early dynamic image data corresponding to the second section based on the identified early section.

9. The method of claim 1, wherein each of the obtained early dynamic image data, the first image data, and the second image data is positron emission tomography (PET) image data.

10. A dynamic image data-based object state prediction device, the device comprising:
a memory; and
at least one processor configured to communicate with the memory,
wherein the processor is configured to:
obtain early dynamic image data corresponding to an early section after a medicine is injected into a learning object until a preset time point;
learn a first prediction model for predicting first image data indicating anatomical information or blood flow information about the learning object corresponding to a time point earlier than a first section by using, as an input, early dynamic image data corresponding to the first section of the early section; and
learn a second prediction model for predicting second image data indicating disease-specific information about the learning object corresponding to a reference time point after the early section by using, as an input, early dynamic image data corresponding to a second section of the early section.

11. The device of claim 10, the processor is configured to:
obtain early dynamic image data corresponding to the first section after the medicine is injected into a diagnostic object; and
predict first image data indicating anatomical information or blood flow information about the diagnostic object corresponding to the earlier time point, by inputting the early dynamic image data corresponding to the first section of the diagnostic object into the learned first prediction model.

12. The device of claim 11, the processor is configured to:
obtain early dynamic image data corresponding to the second section after the medicine is injected into the diagnostic object; and
predict second image data indicating disease-specific information about the diagnostic object corresponding to the reference time point, by inputting the early dynamic image data corresponding to the second section of the diagnostic object into the learned second prediction model.

13. The device of claim 12, the processor is configured to:
when the medicine reduced by a preset amount is injected into the diagnostic object, obtain and process early dynamic image data corresponding to the first section and early dynamic image data corresponding to the second section based on the injection to the diagnostic object, and obtain the processed early dynamic image data corresponding to the first section and the processed early dynamic image data corresponding to the second section; and
input the processed early dynamic image data corresponding to the first section into the first prediction model, and input the processed early dynamic image data corresponding to the second section into the second prediction model.

14. The device of claim 12, wherein an amount of the medicine injected into the learning object is an amount reduced from a reference amount,
wherein the first prediction model is learned based on first label image data corresponding to the first image data,
wherein the second prediction model is learned based on second label image data corresponding to the second image data, and
wherein each of the first label image data and the second label image data is label image data processed according to a medicine injection amount of the learning object.

15. The device of claim 12, where the processor is configured to:
perform spatial normalization before the early dynamic image data corresponding to the first section of the learning object and the diagnostic object is input to the first prediction model; and
perform spatial normalization before the early dynamic image data corresponding to the second section of the learning object and the diagnostic object is input to the second prediction model.
